(19) United States
(12) Patent Application Publication
(10) Pub. No.: US 2017/0059518 A1
(43) Pub. Date: Mar. 2, 2017

(54) SINGLE-USE pH SENSOR STORAGE SOLUTION

(71) Applicant: Rosemount Analytical Inc., Irvine, CA (US)

(72) Inventors: Chang-Dong Feng, Long Beach, CA (US); Hoang Minh Nguyen, Highland, CA (US)

(21) Appl. No.: 15/252,837

(22) Filed: Aug. 31, 2016

Related U.S. Application Data
(60) Provisional application No. 62/212,783, filed on Sep. 1, 2015.

(51) Int. Cl. G01N 27/416; G01N 27/36; C12M 1/34; G01N 27/30

(52) U.S. Cl. CPC G01N 27/4167; G01N 27/302; G01N 27/36; G01N 27/301; G01N 27/4165; C12M 41/26

(57) ABSTRACT

A pH sensor for a single-use bioreactor is provided. The sensor includes a pH sensing electrode, a reference system, a storage compartment, and an access mechanism. The reference system includes a reference electrolyte, a reference electrode disposed in the reference electrolyte, and a reference junction. The storage compartment contains a storage solution that is configured to contact the pH sensing electrode within the storage compartment. The access mechanism is configured to, when actuated, couple the pH sensing electrode to an interior of the single-use bio-reactor. The storage solution includes a buffer solution that is compatible with the reference electrolyte.

### CROSS-REFERENCE TO RELATED APPLICATIONS

The present application is based on and claims the benefit of U.S. Provisional Patent Application Ser. No. 62/212,783 filed Sep. 1, 2015, the content of which is hereby incorporated by reference in its entirety.

### BACKGROUND

Detection and monitoring a system's pH is one of the most common process chemical measurements today. The pH of a solution is a detected measure of relative amounts of hydrogen and hydroxide ions in solution. In fermentation and cell culture processes, one important challenge is to maintain the optimal pH level throughout a reaction. Fermentation processes utilize a live organism, such as a yeast, bacteria, or fungus strain, to produce a desired product. Fermentation processes normally have a relatively short duration (2-7 days). Cell culture, a process in which mammalian cells are grown to produce an active ingredient, typically takes somewhat longer (2-8 weeks).

One challenge for pH measurement in the fermentation and cell culture fields is the cleaning processes required for the fermenter or bioreactor. Specifically, the fermenter or bioreactor must be sterilized prior to use, to ensure against cross-batch contamination or unwanted growths. Such cleaning can include steaming the fermenter or bioreactor as well as the pH sensor. Exposure to high temperatures, steam and rapid thermal shock, can significantly affect a pH sensor's life.

### SUMMARY

A pH sensor for a single-use bioreactor is provided. The sensor includes a pH sensing electrode, a reference system, a storage compartment, and an access mechanism. The reference system includes a reference electrolyte, a reference electrode disposed in the reference electrolyte, and a reference junction. The storage compartment contains a storage solution that is configured to contact the pH sensing electrode within the storage compartment. The access mechanism is configured to, when actuated, couple the pH sensing electrode to an interior of the single-use bio-reactor. The storage solution includes a buffer solution that is compatible with the reference electrolyte.

### BRIEF DESCRIPTION OF THE DRAWINGS

FIG. 1 is a diagrammatic view of a pH sensing bioreactor system with which embodiments of the present invention are particularly useful.

FIGS. 2A-2C provide a plurality of views of a pH sensor with which embodiments of the present invention are particularly useful.

FIGS. 3A-3C provide a plurality of views of a pH sensor and reference system in accordance with one embodiment of the present invention.

FIG. 4 provides an example flow diagram of a method for providing a pH sensor and buffer solution in accordance with one embodiment of the present invention.

FIG. 5 provides an example flow diagram of a method of calibrating and using a pH sensor and buffer solution in accordance with one embodiment of the present invention.

### DETAILED DESCRIPTION OF ILLUSTRATIVE EMBODIMENTS

There exists a need for a disposable pH sensor compatible with plastic bag-type, ready-to-use, disposable bioreactors. Many glass electrode-based pH sensors require that the active surface, or membrane, of the sensor be protected from physical and environmental damage. This function is typically served by a disposable “boot” or cup placed over the sensing end of the sensor.

For many single use bioreactor applications, required sensors are integrated directly onto the bioreactor bag. Once the bioreaction system undergoes sterilization, for example by gamma irradiation, access to the bioreactor interior is prohibited. Therefore, once the bioreactor is in use, access to the pH sensor, for example to provide a new buffer solution, new calibration solution, or to remove a storage solution, is prohibited to reduce the risk of contamination.

Some pH sensors for single-use containers may sit for long periods of time (e.g. multiple years) before they are used. Further, some designs may provide a reference junction in fluidic contact with a storage solution that protects the pH electrode. Since it is possible for at fluid to pass through the reference junction, even if to a miniscule extent, it is important to ensure that such interchange does not materially alter the chemistry of the pH sensor. Further still, there is a need for a storage/calibration solution for a single use pH sensor design. The storage/buffer solution must be compatible with the reference system to allow for minor leakage through the reference junction, and must also be compatible with biologic compounds in the bioreactor.

A pH sensor, such as a glass electrode-based pH sensor, for example, should be kept wet during storage. If allowed to dry out, it can take hours for the electrode to fully re-wet itself, which may be required for a stable pH reading. A glass electrode-based pH sensor may be stored in water. However, it may be beneficial to store the electrode in a pH buffer solution. In at least some embodiments described herein, the storage solution is a pH buffer solution comprising potassium chloride (KCl). For example, a pH sensor with an Ag/AgCl reference system may benefit from a buffer solution with KCl having a known pH in order to facilitate calibration of the sensor prior to use. In some embodiments, a pH sensor undergoes a two-point calibration using a buffer solution prior to use.

In at least some embodiments, the storage solution is discarded into a bioreactor, when the sensor is actuated. Therefore, the buffer solution must be compatible with a biological system of a cell culture process being carried out in a bioreactor. Compatibility, in one embodiment, comprises a buffer solution that, when discharged into the reaction mixture, will not significantly alter an ongoing biological process.

A pH sensor storage solution is desired that is stable enough to last during storage, compatible with a reference system of the pH sensor, and in some instances have a known pH in order to facilitate calibration. At least some embodiments described herein provide a stable, compatible storage solution for a single-use bioreactor pH sensor.

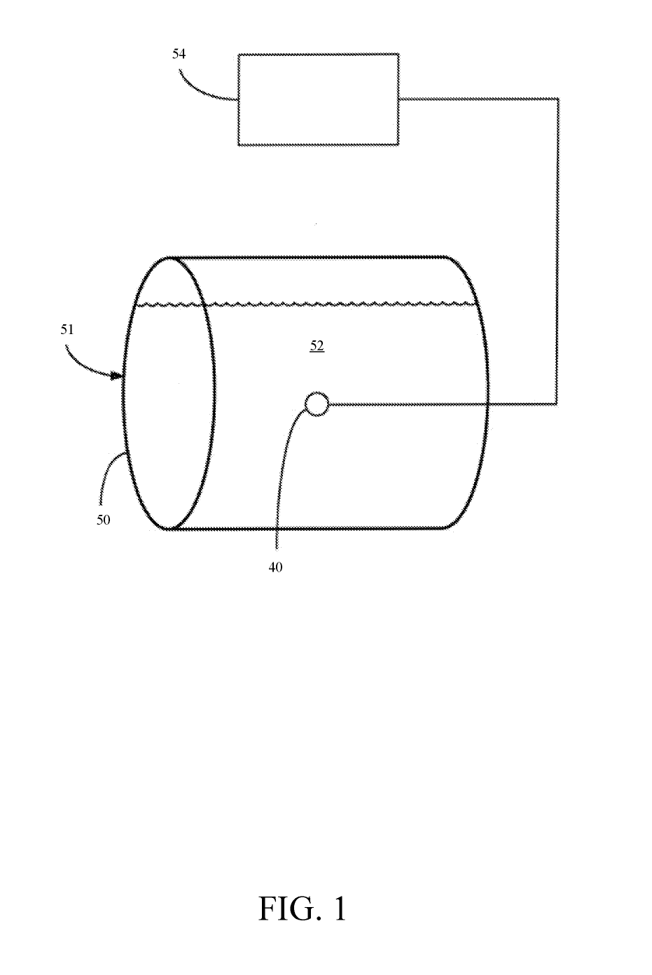

FIG. 1 is a diagrammatic view of a pH sensing bioreactor system with which embodiments of the present invention are particularly useful. pH sensor 40 is electrically coupled to pH analyzer 54, in one embodiment. pH analyzer 54 may be any suitable pH analyzer, or other electrical instrument. pH sensor 40, in one embodiment, is physically attached to a wall 50 of a single use bioreactor/fermenter 51. A sample 52 is disposed within single use bioreactor 50, and is monitored, or otherwise measured, by pH sensor 40.

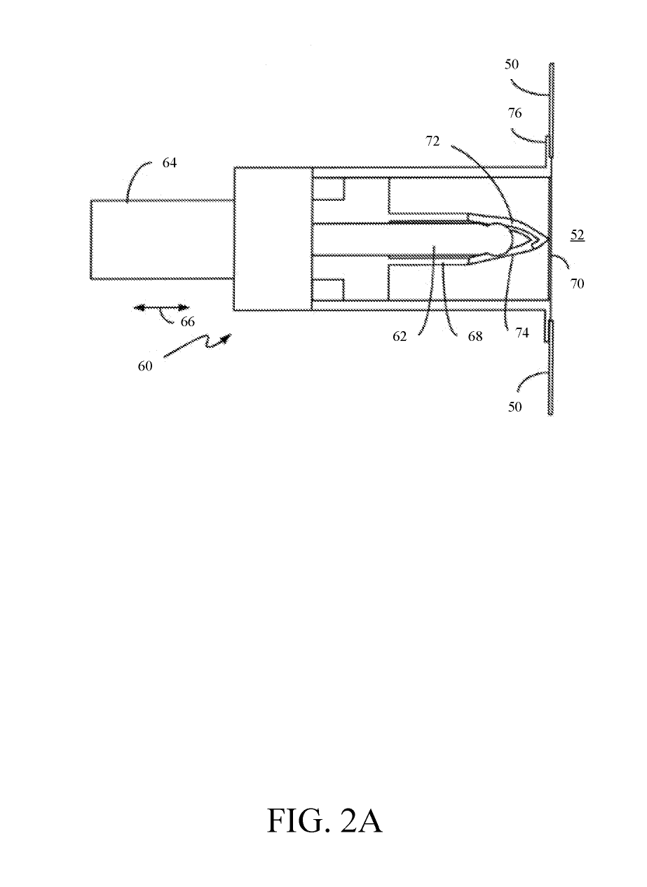

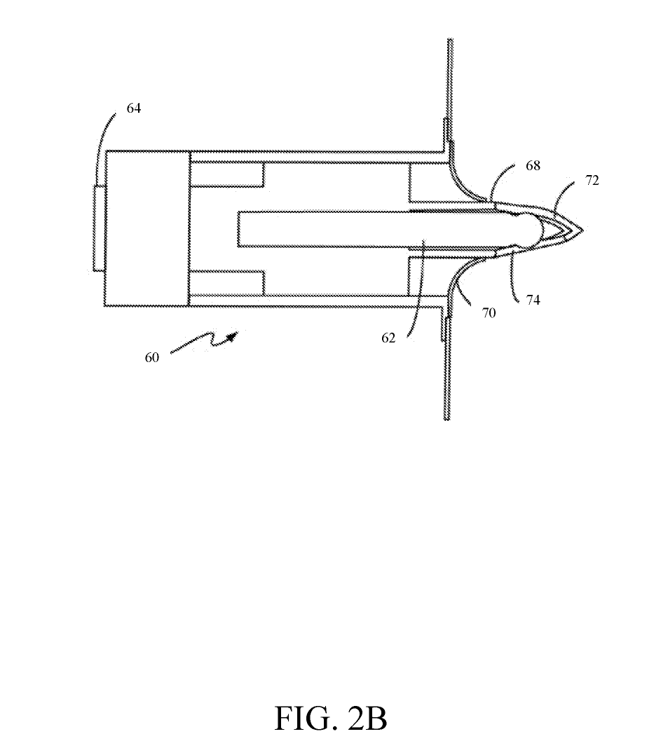

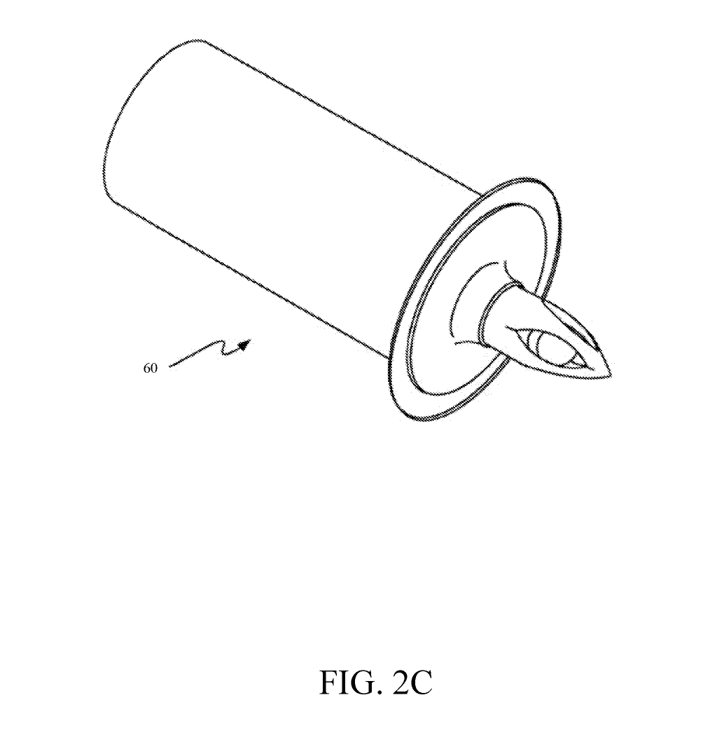

FIGS. 2A-2C provide a number of views of a pH sensor with which embodiments of the present invention are particularly useful. FIG. 2A illustrates a diagrammatic cross-sectional view of a pH sensor 60 illustrated in a “booted” position. The booted position, in one embodiment, for example as shown in FIG. 2A, provides a sensing element, such as electrode 62, separate from, and not in contact with, a sample 52 within a bioreactor. As used herein, a sensing element comprises any electrode, or portion of an electrode, configured to provide an electrical response when exposed to a sample fluid. Accordingly, a sensing element is intended to include glass bulb electrode and a reference junction. pH sensor 60, in one embodiment, includes a plunger 64 that is coupled to an electrode 62 such that axial movement of plunger 64 in the direction indicated at reference numeral 66, will generate corresponding movement of electrode 62.

In one embodiment, pH sensor 60 includes a flange 76 that is fused, adhered, or otherwise bonded to wall 50 of bioreactor 51. In the example shown in FIG. 2A, flange 76 is bonded to an outside surface of wall 50. However, flange 76 could be bonded to an inside surface of wall 50 instead. Flange 76 can be thermally welded, or otherwise permanently attached to sidewall 50 of bioreactor in any suitable manner.

As shown, electrode 62 is disposed within an access spear 68. Access spear 68 is designated as such because it is physically shaped like a spear, in one embodiment, and is configured such that suitable actuation of plunger 64 causes access spear 68 to pierce through membrane 70. In one embodiment, membrane 70 comprises a rubber membrane. When access spear 68 pierces membrane 70, ports 72 and 74, in one embodiment, allow sample 52 to contact electrode 62. When access spear 68 pierces membrane 70, pH sensor 60 is said to be in a service position, for example, shown in FIG. 2B.

FIG. 2C is a diagrammatic perspective view of pH sensor 60 in accordance with one embodiment of the present invention, shown in a service position.

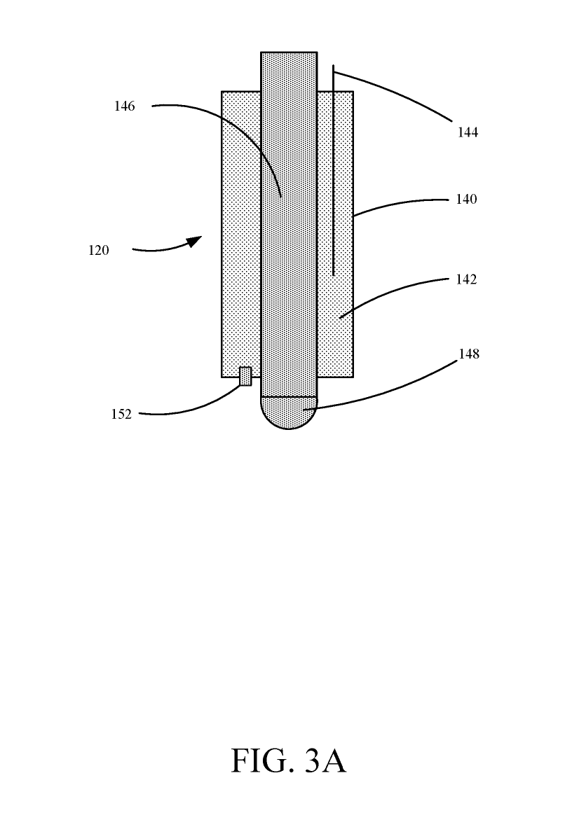

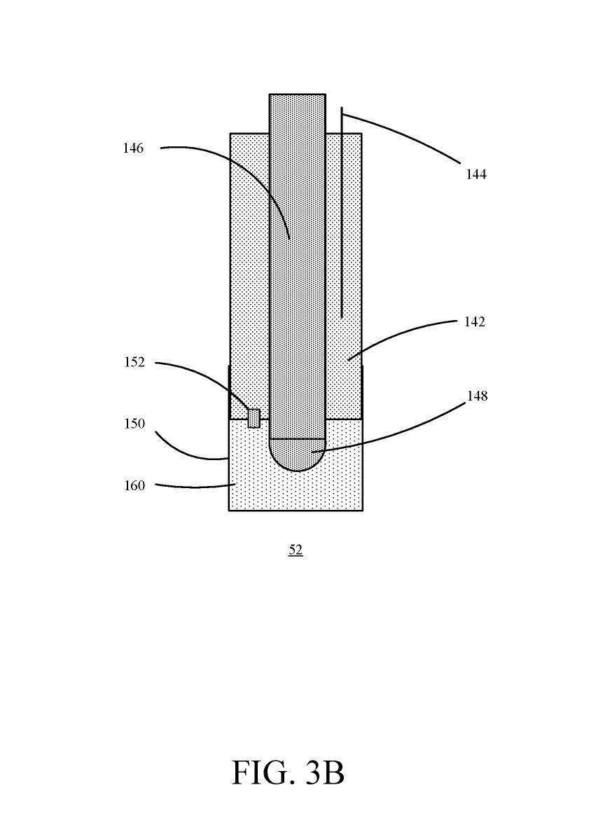

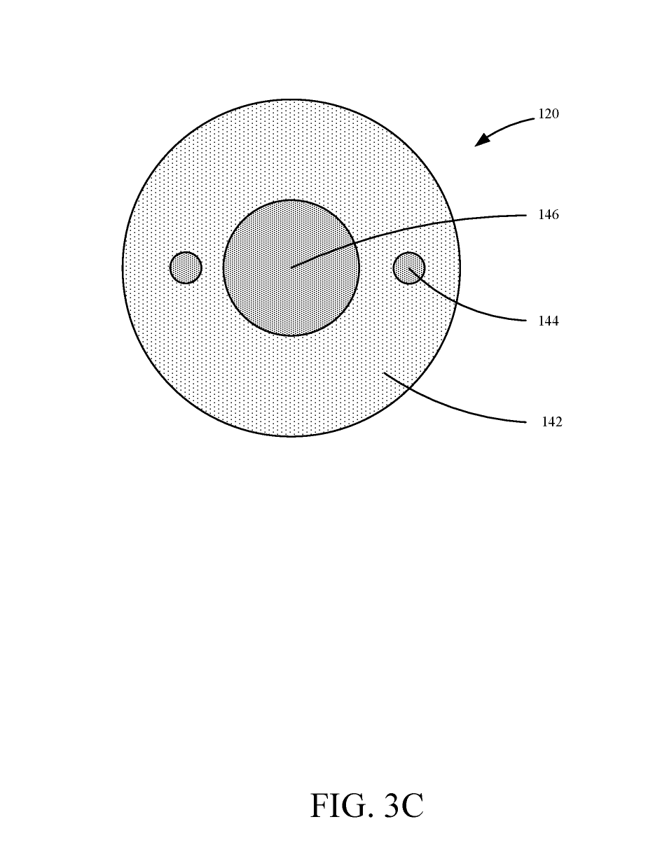

FIGS. 3A-3C provide a different views of a pH sensor and reference system in accordance with one embodiment of the present invention. FIG. 3A is a cross-sectional view of a pH sensor 120. Sensor 120 may include, or couple to a flange/support (not shown in FIG. 3A), in one embodiment, which may couple sensor 120 to a wall of a single use bioreactor/mixer. pH sensor 120 also comprises a sensor body 140 which contains a suitable reference electrolyte 142, and reference electrode 144. In one embodiment, reference electrode 144 is a silver electrode, in an Ag/AgCl reference system, for example. Additionally, in one embodiment, sensing element (glass electrode) 146 is disposed, at least partially, within sensor body 140 and extends such that distal sensing portion 148 is disposed within storage chamber 150 when sensor 120 is in the booted position, for example as illustrated in FIG. 3B. Additionally, a sensing element, such as reference junction 152, is physically isolated from storage chamber 150.

Sensor 120 may transition between a storage and a sensing position. In a first position, for example that shown in FIG. 3B, the sensor is in a booted position, such that sensing portion 148 is protected from damage, and in contact with a storage solution 160 within storage chamber 150.

FIG. 3B is a diagrammatic view of pH sensor 120 arranged in a storage position, exposed to a storage solution. As set forth above, in some embodiments, the pH of the storage solution may be known and thus can be used for calibration of the pH sensor. In the position shown in FIG. 3B, reference junction 152 is in fluidic communication with sensing portion 148 of sensing electrode 146. Additionally, in one embodiment, storage chamber 150 is fluidically isolated from sample 52 by a physical barrier, such as storage chamber 150. Given that the storage solution within storage chamber 150 can be provided having a precisely known pH, sensor 120 can be calibrated to ensure that its output corresponds with the known pH of the storage solution. In one embodiment, the buffer solution is configured to be compatible with a reference system. In one embodiment, the buffer solution is configured for compatibility with an Ag/AgCl/KCl system.

FIG. 3C illustrates a top view of sensor 120 showing one example configuration of sensing electrode 146 within sensor body 140.

In one embodiment, the pH sensor described herein employs an Ag/AgCl/KCl reference system. In such embodiment, the storage solution is selected to be compatible to the reference system. In particular, the storage solution will be a buffered solution of potassium chloride. The buffer helps reduce or eliminate changes in pH that may occur as the storage solution leaks into the reference electrolyte through the reference junction, or vice versa. The buffer used for the storage solution can be any suitable buffer, but is preferably one of a carbonate-based buffer and a phosphate-based buffer. In some embodiments, the pH of the storage solution may be known (e.g. tested during manufacture and written on the product packaging) and used to calibrate the pH sensor prior to use. While an Ag/AgCl/KCl reference system is described above, it is expressly contemplated that embodiments of the present invention are applicable to other types of reference systems.

In one embodiment, the pH sensor includes an Ag/AgCl/KCl reference system and a phosphate-based buffer solution that shares an electrolyte with the reference system. In another embodiment, a carbonate-based buffer solution is provided instead of the phosphate-based buffer solution. In both examples, the buffer solution is compatible with the reference system of the pH electrode.

In one embodiment, the buffer solution comprises a high concentration of KCl. For example, the concentration of KCl is at least 0.05 M with respect to saturation of KCl solution. In other embodiments, the concentration of KCl can be higher, such as at least 0.06 M, at least 0.07M, at least 0.08M, 0.09 M, and 0.10M. In one embodiment, the concentration of KCl comprises a saturation point of KCl for the system, calculated based on known saturation points.

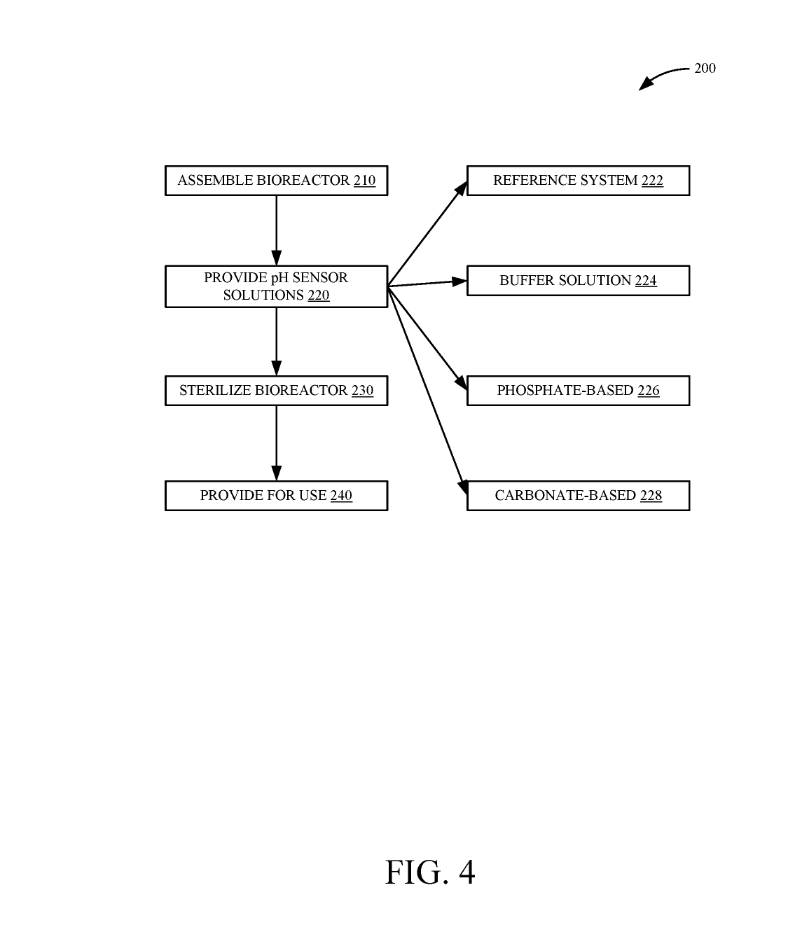

FIG. 4 provides an example flow diagram of a method for providing a pH sensor and buffer solution in accordance with one embodiment of the present invention. Method 200 can be used, for example, with either of the reactors presented in FIGS. 2A-2C or FIGS. 3A-3C. Method 200 may also be useful, for example, with other appropriate single-use bioreactor configurations. Further, method 200 may be useful for pH sensors with a single storage compartment, or with pH sensors having a calibration solution separate from a storage solution.

In block 210, a bioreactor is assembled by providing a single use bioreactor with one or more sensors for a given bioreaction. For example, temperature and/or pressure sensors may be provided in addition to a pH sensor, depending on anticipated reaction conditions and associated monitoring requirements. Assembling the bioreactor, in block 210 comprises attaching sufficient sensor ports and sensors to a surface of the bioreactor to accommodate all desired sensor connections. In one embodiment, all desired sensor ports must be attached to the bioreactor bag prior to a sterilization process in order to reduce the risk of sample contamination.

In block 220, pH sensor solutions are provided to a pH sensor. In one embodiment, providing a pH sensor solution to a pH sensor occurs, chronologically, prior to the pH sensor being attached to the bioreactor. In another embodiment, providing a pH sensor solution occurs post- attachment of the pH sensor to the bioreactor bag.

In one embodiment, providing a pH sensor solution, as indicated in block 222, comprises providing a reference solution for a reference system for calibration of the pH sensor. In one embodiment, the reference system is an Ag/AgCl/KCl system. In one embodiment, providing a pH sensor solution, as indicated in block 224, comprises providing a pH buffer solution configured to maintain wetting of the pH sensor electrode during storage, and to facilitate calibration. In one embodiment, providing a pH sensor solution comprises providing a phosphate-based pH sensor solution, as indicated in block 226. In another embodiment, providing a pH sensor solution comprises providing a carbonate-based pH sensor solution, as indicated in block 228. In one embodiment, both the reference solution and the buffer solution are the same, for example either both phosphate-based, or both carbonate-based, solutions. In another embodiment, reference solution and buffer solution are different, for example one phosphate-based solution and one carbonate-based solution.

In one embodiment, method 200 comprises providing a pH sensor with a buffer solution compatible with an Ag/AgCl/KCl reference system. In one embodiment, method 200 comprises providing a pH sensor with a pH buffer solution compatible with a biological system ongoing within the bioreactor. As shown, method 200 can include providing a phosphate buffer solution or a carbonate buffer solution. However, the concentration of the buffer solution is relatively high, such in the range of at least 0.05M to at least 0.10M KCl.

In block 230, the bioreactor system undergoes a sterilization procedure. In one embodiment, sterilization comprises the bioreactor bag, and attached sensor ports and components, undergoing gamma irradiation. In one embodiment, post-sterilization, sensors cannot be removed or newly attached to the bioreactor bag, in order to reduce the risk of contamination to the bioreactor system.

In block 240, the sterilized bioreactor system, including coupled sensors, is provided for use. In one embodiment, providing a bioreactor system comprises providing a bioreactor with a pH sensor ready for deployment. As set forth above the bioreactor system may include a pH sensor with a buffer solution that is compatible with a biological system. Compatibility with a biological system, in one embodiment, comprises a buffer solution that will not substantially alter ongoing reaction conditions within a bioreactor. In another embodiment, a pH buffer solution compatible with a biological system comprises a pH buffer solution that does not contain any materials toxic or otherwise harmful to a biological compound within the bioreactor. In another embodiment, providing a pH buffer compatible with a biological system comprises a pH buffer composed substantially of inert compounds that will not react with either reactants or products of a bioreaction system.

The pH sensor provided in block 240, in one embodiment, comprises a buffer solution compatible with the reference system. For example, the buffer solution may be compatible with an Ag/AgCl reference system. In one embodiment, method 200 includes providing a pH sensor with a stable buffer solution configured to be stable for at least one year or more.

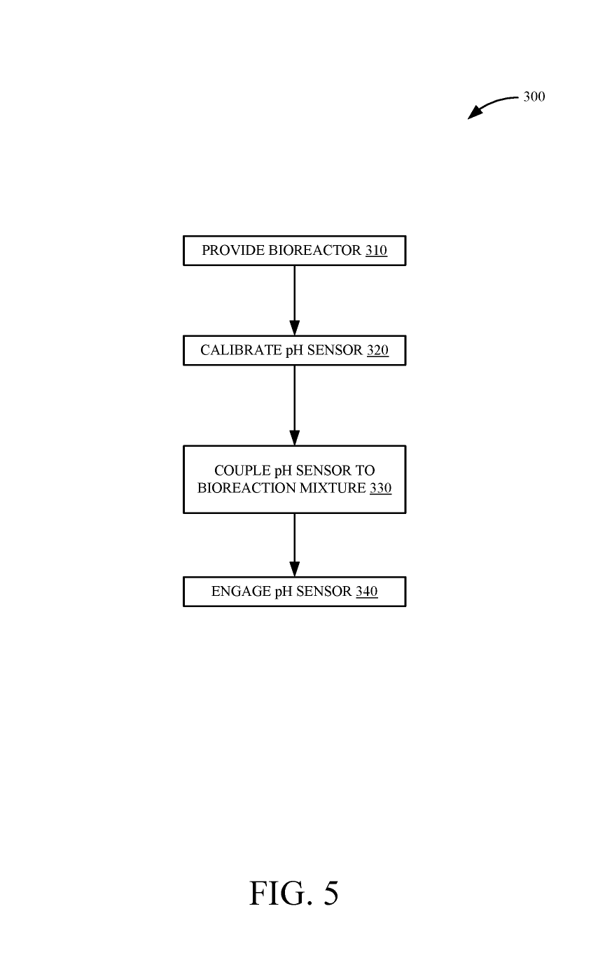

FIG. 5 is a flow diagram of a method of calibrating and using a pH sensor and buffer solution in accordance with one embodiment of the present invention. Method 300 may be useful to calibrate a pH sensor, and detect a solution pH of an ongoing bioreaction within a single use bioreactor, for example, a bioreactor configuration such as that in FIGS. 2A-2C or FIGS. 3A-3C, or another suitable single use bioreactor coupled to a pH sensor.

In block 310, a bioreactor is provided. In one embodiment, providing a bioreactor comprises providing a bioreactor with a pH sensor that has previously undergone a sterilization process. In another embodiment, providing a bioreactor comprises providing an unsterilized bioreactor with pH sensor configured to undergo a sterilization process, for example gamma irradiation, once all desired sensors and sensor components are attached.

In block 320, the pH sensor is calibrated. Calibration may comprise comparing a known pH of the storage solution to that reported by a pH analyzer coupled to the pH sensor. In one embodiment, this may comprise a transition of the pH sensor, within a pH sensor housing, from a storage to calibration position.

In block 330, the pH sensor engages a bioreaction mixture. In one embodiment, a single use bioreactor with pH sensor is configured to be used only once, such that once the pH sensor engages the bioreaction mixture, it may not be used again. In one embodiment, providing a pH sensor in engaged with a bioreaction system includes at least a partial capture of the storage solution, for example within a storage receptacle of a pH sensor. In another embodiment, substantially all of the storage solution mixes with the bioreaction mixture when the pH sensor is actuated.

In block 340, a pH sensor, when actuated, engages a bioreaction system. A pH sensor may be configured, in one embodiment, to provide ongoing pH measurements at the beginning, during, or at the end of a bioreaction system.

Method 300 may include the use of a pH buffer solution configured to be compatible with a reference system of the pH sensor. In one embodiment, the reference system is an Ag/AgCl/KCl system. The buffer solution can be configured to be sufficiently stable to maintain a quality of the pH electrode during a storage period of at least one year. Method 300 can be configured for use with a pH sensor with pH buffer compatible with an intended biological system of a bioreactor, such that discharge of the pH buffer into the bioreaction mixture does not have a substantial effect on the ongoing bioreaction process.

In one embodiment, the pH sensor used in method 300 includes a phosphate-based buffer solution or a carbonate-based buffer solution. The pH sensor used in method 300 may include a pH buffer solution with a high concentration of KCl. In one embodiment, a high concentration comprises an at least 0.05 M KCl solution.

Although the present invention has been described with reference to preferred embodiments, workers skilled in the art will recognize that changes may be made in form and detail without departing from the spirit and scope of the invention.

What is claimed is:

1. A pH sensor for a single-use bioreactor, the sensor comprising:
a pH sensing electrode;
a reference system including a reference electrolyte, a reference electrode disposed in the reference electrolyte, and a reference junction;
a storage compartment containing a storage solution, wherein the storage solution is configured to contact the pH sensing electrode within the storage compartment;
an access mechanism configured to, when actuated, couple the pH sensing electrode to an interior of the single-use bioreactor; and
wherein the storage solution comprises a buffer solution that is compatible with the reference electrolyte.

2. The pH sensor of claim 1, wherein the reference system is a a silver/silver-chloride reference system and wherein the reference electrolyte is potassium chloride.

3. The pH sensor of claim 1, wherein the storage solution comprises a phosphate-based buffer solution.

4. The pH sensor of claim 1, wherein the storage solution comprises a carbonate-based buffer solution.

5. The pH sensor of claim 1, wherein the storage solution comprises a substantially 0.10 molar potassium chloride solution.

6. The pH sensor of claim 1, wherein the storage solution has a known pH to calibrate the pH sensor prior to actuation of the access mechanism.

7. A pH sensor comprising:
a sensing electrode configured to detect a pH of a sample solution;
a reference system including a reference electrolyte, a reference electrode disposed in the reference electrolyte, and a reference junction;
a storage compartment configured to house the sensing electrode prior to deployment, the storage compartment being fluidically coupled to the reference junction; and
a storage solution within the storage compartment, configured to contact the sensing electrode, wherein the storage solution comprises a buffered solution that is configured to allow fluid leakage through the reference junction without changing a pH of the storage solution.

8. The pH sensor of claim 7, wherein the sample solution comprises live microorganisms, and wherein storage solution is configured to be substantially inert with regard to the sample solution.

9. The pH sensor of claim 7, wherein the storage solution has a known pH.

10. The pH sensor of claim 9, wherein the reference system comprises a silver/silver chloride reference system and the reference electrolyte includes a potassium chloride solution.

11. The pH sensor of claim 7, wherein the storage solution comprises a phosphate-based buffer.

12. The pH sensor of claim 7, wherein the storage solution comprises a carbonate-based buffer.

13. The pH sensor of claim 7, wherein the storage solution comprises a saturated potassium chloride solution.

14. The pH sensor of claim 7, wherein the storage solution comprises a substantially 0.10 molar potassium chloride solution.

15. The pH sensor of claim 7, wherein the pH sensor is configured to be physically coupled to a single-use bioreaction chamber.

16. A method for measuring pH in a single-use bioreaction chamber, the method comprising:
coupling a pH sensor to the single-use bioreaction chamber, wherein the pH sensor comprises a storage compartment housing a pH-sensing electrode within a buffer solution comprising potassium chloride, wherein coupling comprises positioning the pH sensing-electrode in proximity to, but not in physical contact with, an interior of the single-use bioreaction chamber;
calibrating the pH sensor;
contacting the pH sensor to a solution within the interior of the single-use bioreaction chamber; and
detecting a pH of the solution.

17. The method of claim 16, wherein calibrating the pH sensor comprises measuring a response of the pH sensor to the buffer solution where the buffer solution has a known pH.

18. The method of claim 16, wherein contacting the pH sensor to the solution comprises breaching a wall of the bioreaction chamber such that a portion of the buffer solution is discharged into the bioreaction chamber, and wherein the buffer solution is substantially inert with respect to the solution.

19. The method of claim 16, wherein the buffer solution comprises a phosphate-based buffer solution.

20. The method of claim 16, wherein the buffer solution comprises a carbonate-based buffer solution.
